(19) United States
(12) Patent Application Publication
(10) Pub. No.: US 2007/0225304 A1
(43) Pub. Date: Sep. 27, 2007

(54) AMINOPURINE DERIVATIVES FOR TREATING NEURODEGENERATIVE DISEASES

(75) Inventors: Andrew G. Cole, Robbinsville, NJ (US); Marc-Raleigh Brescia, Dayton, NJ (US); Gulzar Ahmed, Voorhees, NJ (US); Ian Henderson, Hopewell, NJ (US)

Correspondence Address: HESLIN ROTHENBERG FARLEY & MESITI PC, 5 COLUMBIA CIRCLE, ALBANY, NY, 12203, US

(73) Assignee: Pharmacopeia Drug Discovery, Inc., Cranbury, NJ (US)

(21) Appl. No.: 11/470,347

(22) Filed: Sep. 6, 2006

Related U.S. Application Data
(60) Provisional application No. 60/714,449, filed on Sep. 6, 2005.

(51) Int. Cl. A61K 31/52; C07D 473/12

(52) U.S. Cl. 514/263.22; 514/263.23; 514/263.4; 544/277

(57) ABSTRACT

The invention relates to aminopurine derivatives useful in treating disorders that are mediated by adenosine receptor function, including neurodegenerative diseases and inflammation. The compounds are of the general formula:

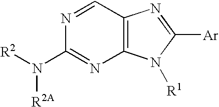

### CROSS REFERENCE TO RELATED APPLICATIONS

This application claims benefit from U.S. Provisional Application 60/714,449, filed on Sep. 6, 2005, the entire contents of which are incorporated herein by reference.

### FIELD OF THE INVENTION

The invention relates to substituted aminopurine derivatives useful in treating disorders that are mediated by adenosine receptor function, including neurodegenerative diseases and inflammation.

### BACKGROUND OF THE INVENTION

Adenosine is a modulator of multiple physiological functions, including cardiovascular, neurological, respiratory and renal fuinctions. Adenosine mediates its effects through specific G-protein coupled receptors A₁, A₂ₐ, A_{2b} and A₃.

Adenosine 2a (A₂ₐ) receptor antagonists useful in the treatment of Parkinson's disease have been disclosed in U.S. Pat. No. 6,875,772 and U.S. Pat. No. 6,787,541. A2ₐ antagonists have also been shown to be useful for the treatment of restless leg syndrome (as outlined in WO 2004019949). These disclosures are incorporated herein by reference as they relate to utility.

### SUMMARY OF THE INVENTION

In one aspect the present invention provides compounds according to formula I, useful as adenosine 2a (A2a) receptor antagonists:

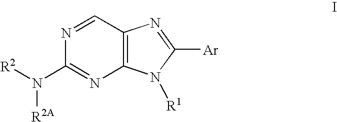

In these compounds
R¹ is a C₃-C₂₀ hydrocarbon in which at least one —CH₂— has been replaced by —O—,
R² is selected from the group consisting of H and lower alkyl;
R^{2A} is selected from the group consisting of C₁-C₂₀ hydrocarbon, heterocyclyl, heterocyclylalkyl, substituted alkyl, substituted arylaLkyl and substituted heterocyclylalkyl;
Ar represents aryl, heteroaryl, substituted aryl and substituted heteroaryl.

In another aspect, the invention relates to pharmaceutical compositions comprising a therapeutically effective amount of at least one compound of general formula I or a pharmaceutically acceptable salt thereof; and a pharmaceutically acceptable carrier.

The compounds and pharmaceutical compositions described herein are useful in methods for preventing and treating a condition for which an antagonist of adenosine 2a receptor is indicated.

In a third aspect, the invention relates to a method for treating a disease by antagonizing a response mediated by adenosine 2a receptors. The method comprises bringing into contact with adenosine receptor at least one compound of general formula I or a pharmaceutically acceptable salt thereof.

In yet another aspect the present invention relates to a method of treating disease mediated by adenosine receptors in a subject in need thereof comprising administering to the subject a therapeutically effective amount of at least one compound of general formula I or a pharmaceutically acceptable salt thereof. Treating a disorder associated with adenosine receptor function includes treating disorders associated with A₂ₐ receptors and one or more additional adenosine receptors, such as A₁, A_{2b} or A₃ receptors.

The compounds of the present invention are useful in effecting neuroprotection and as such the present invention provides a method of neuroprotection in a subject in need thereof comprising administering to the subject a therapeutically effective amount of at least one compound of general formula I or a pharmaceutically acceptable salt thereof.

Other indications in which the adenosine antagonists are useful include central nervous system disorders, neurodegenerative diseases, cardiovascular disorders, and diabetes.

The compounds of the present invention are useful in stand alone treatments or in combination with one or more of (1) an agent useful in the treatment of Parkinson's disease, e.g L-dopa, caffeine or other dopaminergic receptor agonist (2) an agent useful in the treatment of movement disorders, (3) an agent useful in the treatment of depression.

### DETAILED DESCRIPTION OF THE INVENTION

Throughout this specification the substituents are defined when introduced and retain their definitions.

It has now been found that compounds of general formula I are potent antagonists of the adenosine 2a (A₂ₐ) receptor:

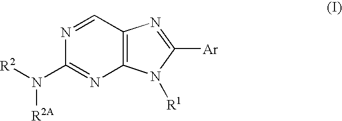

In these compounds R¹ is a C₃-C₂₀ hydrocarbon in which at least one —CH₂— has been replaced by —O—. In many embodiments R¹ is selected from the group consisting of alkoxyalkyl, alkoxyaryl, alkoxyarylalkyl and oxygen heterocycles. For example, R¹ may be alkoxyalkyl, such as methoxypropyl, or alkoxyphenyl, such as methoxyphenyl or oxygen heterocycle, such as tetrahydropyran.

In certain embodiments R² is H or methyl.

In certain embodiments R^{2A} is C₁-C₂₀ hydrocarbon. In others, R^{2A} gives rise to compounds of formula 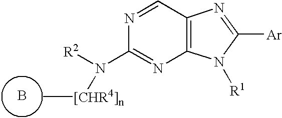
in which B is an aryl or heteroaryl ring, optionally substituted; R⁴ is H or methyl; and n is 1 to 4. In these compounds R⁴ can be, in each of its occurrences independently H or methyl. For example, when n is 2, the chain between N and B can be any of —CH₂CH₂—, —CH(CH₃)CH₂—, —CH₂CH(CH₃)— or —CH(CH₃)CH(CH₃)—. In certain embodiments n is 1 or 2, and B may be chosen from phenyl; phenyl substituted with halogen, methoxy, methyl or trifluoromethyl; thienyl, furanyl and pyridinyl.

In some embodiments R^{2A} is selected from benzyl and substituted benzyl. In some embodiments R^{2A} is selected from chlorobenzyl, fluorobenzyl and methoxybenzyl. In other embodiments R^{2A} is selected from phenylethyl and substituted phenylethyl. In some embodiments R^{2A} is selected from chlorophenylethyl, fluorophenylethyl, phenylmethylethyl and methoxyphenylethyl. In some embodiments R^{2A} is heteroarylalkyl. In some embodiments R^{2A} is selected from thienylmethyl, thienylmethyl, and pyridinylethyl. In some embodiments R^{2A} is heterocyclyl, for example a 5- or 6-membered heterocycle, such as piperidine or morpholine. In certain embodiments R^{2A} is selected from lower alkyl and carbocyle.

In some embodiments, Ar is phenyl; thienyl; furanyl; or phenyl substituted with cyano, halogen, methoxy, methyl or trifluoromethyl. When Ar is phenyl or substituted phenyl, the compounds have the chemical formula as shown below:

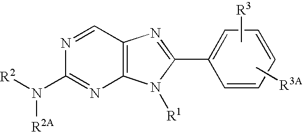

In certain embodiments, R³ and R^{3A} may be independently H, CN or halogen.

In some embodiments R¹ is methoxypropyl; R^{2A} is selected from aryl, arylalkyl, substituted aryl and substituted arylalkyl and Ar is selected from phenyl and substituted phenyl, having chemical formula as shown below:

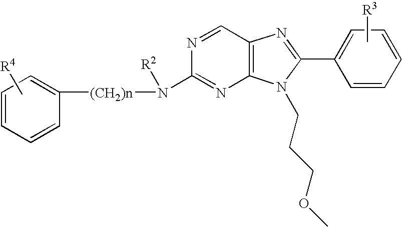

In another aspect the present invention provides a method of treating a disorder, which is mediated by adenosine 2a (A₂ₐ) receptor function, which comprises administering to a subject in need of such treatment a therapeutically effective amount of a compound of formula I. It also encompasses a method of treating a disorder associated with A₂ₐ receptor and one or more of A₁, A_{2b} or A₃ receptors. All of the compounds falling within the foregoing parent genera and their subgenera are useful as adenosine receptor antagonists.

For convenience and clarity certain terms employed in the specification, examples and claims are described herein.

Alkyl is intended to include linear, branched, or cyclic hydrocarbon structures and combinations thereof. Lower alkyl refers to alkyl groups of from 1 to 6 carbon atoms. Examples of lower alkyl groups include methyl, ethyl, propyl, isopropyl, butyl, s- and t-butyl and the like. Preferred alkyl groups are those of C₂₀ or below. Cycloalkyl is a subset of alkyl and includes cyclic hydrocarbon groups of from 3 to 8 carbon atoms. Examples of cycloalkyl groups include c-propyl, c-butyl, c-pentyl, norbornyl and the like.

C₁ to C₂₀ hydrocarbon includes alkyl, cycloalkyl, alkenyl, alkynyl, aryl and combinations thereof. Examples include phenethyl, cyclohexylmethyl, camphoryl, adamantyl and naphthylethyl.

Alkoxy or alkoxyl refers to groups of from 1 to 8 carbon atoms of a straight, branched, cyclic configuration and combinations thereof attached to the parent structure through an oxygen. Examples include methoxy, ethoxy, propoxy, isopropoxy, cyclopropyloxy, cyclohexyloxy and the like. Lower-alkoxy refers to groups containing one to four carbons. When used to describe a substituent on an aryl ring, alkoxy also is intended to encompass methylene dioxy.

Alkoxyalkyl refers to ether groups of from 3 to 8 atoms of a straight, branched, cyclic configuration and combinations thereof attached to the parent structure through an alkyl. Examples include methoxymethyl, methoxyethyl, ethoxypropyl, and the like.

Alkoxyaryl refers to alkoxy substituents attached to an aryl, wherein the aryl is attached to the parent structure.

Acyl refers to groups of from 1 to 8 carbon atoms of a straight, branched, cyclic configuration, saturated, unsaturated and aromatic and combinations thereof, attached to the parent structure through a carbonyl functionality. One or more carbons in the acyl residue may be replaced by nitrogen, oxygen or sulfur as long as the point of attachment to the parent remains at the carbonyl. Examples include acetyl, benzoyl, propionyl, isobutyryl, t-butoxycarbonyl, benzyloxycarbonyl and the like. Lower-acyl refers to groups containing one to four carbons.

Aryl and heteroaryl mean a 5- or 6-membered aromatic or heteroaromatic ring containing 0-3 heteroatoms selected from O, N, or S; a bicyclic 9- or 10-membered aromatic or heteroaromatic ring system containing 0-3 heteroatoms selected from O, N, or S; or a tricyclic 13- or 14-membered aromatic or heteroaromatic ring system containing 0-3 heteroatoms selected from O, N, or S. The aromatic 6- to 14-membered carbocyclic rings include, e.g., benzene and naphthalene, and according to the invention benzoxalane and residues in which one or more rings are aromatic, but not all need be.

The 5- to 10-membered aromatic heterocyclic rings include, e.g., imidazole, pyridine, indole, thiophene, benzopyranone, thiazole, furan, benzimidazole, quinoline, isoquinoline, quinoxaline, pyrimidine, pyrazine, tetrazole and pyrazole.

Arylalkyl refers to a substituent in which an aryl residue is attached to the parent structure through alkyl. Examples are benzyl, phenethyl and the like. Heteroarylalkyl refers to a substituent in which a heteroaryl residue is attached to the parent structure through alkyl. Examples include, e.g., pyridinylmethyl, pyrimidinylethyl and the like.

Heterocycle means a cycloalkyl or aryl residue in which from one to three carbons is replaced by a heteroatom selected from the group consisting of N, O and S. The nitrogen and sulfur heteroatoms may optionally be oxidized, and the nitrogen heteroatom may optionally be quaternized. Examples of heterocycles include pyrrolidine, pyrazole, pyrrole, indole, quinoline, isoquinoline, tetrahydroisoquinoline, benzofuran, benzodioxan, benzodioxole (commonly referred to as methylenedioxyphenyl, when occurring as a substituent), tetrazole, morpholine, thiazole, pyridine, pyridazine, pyrimidine, thiophene, furan, oxazole, oxazoline, isoxazole, dioxane, tetrahydrofuran and the like. It is to be noted that heteroaryl is a subset of heterocycle in which the heterocycle is aromatic. According to convention, the suffix “yl” indicates the moiety in question appearing as a residue on a parent structure. Thus, for example, heterocyclyl means a heterocycle appearing as a substituent rather than a parent. Examples of heterocyclyl residues additionally include piperazinyl, 2-oxopiperazinyl, 2-oxopiperidinyl, 2-oxo-pyrrolidinyl, 2-oxoazepinyl, azepinyl, 4-piperidinyl, pyrazolidinyl, imidazolyl, imidazolinyl, imidazolidinyl, pyrazinyl, oxazolidinyl, isoxazolidinyl, thiazolidinyl, isothiazolyl, quinuclidinyl, isothiazolidinyl, benzimidazolyl, thiadiazolyl, benzopyranyl, benzothiazolyl, tetrahydrofuryl, tetrahydropyranyl, thienyl, benzothienyl, thiamorpholinyl, thiamorpholinylsulfoxide, thiamorpholinylsulfone, oxadiazolyl, triazolyl and tetrahydroquinolinyl.

An oxygen heterocycle is a heterocycle containing at least one oxygen in the ring; it may contain additional oxygens, as well as other heteroatoms. A sulfur heterocycle is a heterocycle containing at least one sulfur in the ring; it may contain additional sulfurs, as well as other heteroatoms. A nitrogen heterocycle is a heterocycle containing at least one nitrogen in the ring; it may contain additional nitrogens, as well as other heteroatoms. Oxygen heteroaryl is a subset of oxygen heterocycle; examples include furan and oxazole. Sulfur heteroaryl is a subset of sulfur heterocycle; examples include thiophene and thiazine. Nitrogen heteroaryl is a subset of nitrogen heterocycle; examples include pyrrole, pyridine and pyrazine.

Substituted alkyl, aryl, cycloalkyl, heterocyclyl etc. refer to alkyl, aryl, cycloalkyl, or heterocyclyl wherein up to three H atoms in each residue are replaced with halogen, haloalkyl, hydroxy, loweralkoxy, carboxy, carboalkoxy (also referred to as alkoxycarbonyl), carboxamido (also referred to as alkylaminocarbonyl), cyano, carbonyl, nitro, amino, alkylamino, dialkylamino, mercapto, alkylthio, sulfoxide, sulfone, acylamino, amidino, phenyl, benzyl, heteroaryl, phenoxy, benzyloxy, or heteroaryloxy.

The terms “halogen” and “halo” refer to fluorine, chlorine, bromine or iodine.

Some of the compounds described herein may contain one or more asymmetric centers and may thus give rise to enantiomers, diastereomers, and other stereoisomeric forms that may be defmed, in terms of absolute stereochemistry, as (R)- or (S)-. The present invention is meant to include all such possible isomers, as well as, their racemic and optically pure forms. Optically active (R)- and (S)-isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. Likewise, all tautomeric forms are also intended to be included. The configuration of any carbon-carbon double bond appearing herein is selected for convenience only and is not intended to designate a particular configuration; thus a carbon-carbon double bond depicted arbitrarily herein as trans may be Z, E or a mixture of the two in any proportion.

The graphic representations of racemic, ambiscalemic and scalemic or enantiomerically pure compounds used herein are taken from Maehr, J. Chem. Ed. 62, 114-120 (1985): solid and broken wedges are used to denote the absolute configuration of a chiral element; wavy lines indicate disavowal of any stereochemical implication which the bond it represents could generate; solid and broken bold lines are geometric descriptors indicating the relative configuration shown but denoting racemic character; and wedge outlines and dotted or broken lines denote enantiomerically pure compounds of indeterminate absolute configuration.

It will be recognized that the compounds of this invention can exist in radiolabeled form, i.e., the compounds may contain one or more atoms containing an atomic mass or mass number different from the atomic mass or mass number usually found in nature. Radioisotopes of hydrogen, carbon, phosphorous, fluorine, chlorine and iodine include ³H, ¹⁴C, ³⁵S, ¹⁸F, ³⁶Cl and ¹²⁵I, respectively. Compounds that contain those radioisotopes and/or other radioisotopes of other atoms are within the scope of this invention. Tritiated, i.e. ³H, and carbon-14, i.e., ¹⁴C, radioisotopes are particularly preferred for their ease in preparation and detectability. Radiolabeled compounds of this invention can generally be prepared by methods well known to those skilled in the art. Conveniently, such radiolabeled compounds can be prepared by carrying out the procedures disclosed in the Examples by substituting a readily available radiolabeled reagent for a non-radiolabeled reagent. Because of the high affinity for the A2a receptor, radiolabeled compounds of the invention are useful for A2a receptor assays.

Terminology related to “protecting”, “deprotecting” and “protected” functionalities occurs throughout this application. Such terminology is well understood by persons of skill in the art and is used in the context of processes that involve sequential treatment with a series of reagents. In that context, a protecting group refers to a group which is used to mask a functionality during a process step in which it would otherwise react, but in which reaction is undesirable. The protecting group prevents reaction at that step, but may be subsequently removed to expose the original functionality. The removal or “deprotection” occurs after the completion of the reaction or reactions in which the functionality would interfere. Thus, when a sequence of reagents is specified, as it is in the processes of the invention, the person of ordinary skill can readily envision those groups that would be suitable as “protecting groups”. Suitable groups for that purpose are discussed in standard textbooks in the field of chemistry, such as Protective Groups in Organic Synthesis by T. W. Greene [John Wiley & Sons, New York, 1991], which is incorporated herein by reference.

A comprehensive list of abbreviations utilized by organic chemists appears in the first issue of each volume of the Journal of Organic Chemistry. The list, which is typically presented in a table entitled “Standard List of Abbreviations”, is incorporated herein by reference.

In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants that are in themselves known, but are not mentioned here. The starting materials, for example in the case of suitably substituted benzimidazole ring compounds, are either commercially available, synthesized as described in the examples or may be obtained by the methods well known to persons of skill in the art.

The present invention further provides pharmaceutical compositions comprising as active agents, the compounds described herein.

As used herein a “pharmaceutical composition” refers to a preparation of one or more of the compounds described herein, or physiologically acceptable salts or solvents thereof, with other chemical components such as physiologically suitable carriers and excipients.

Pharmaceutical compositions for use in accordance with the present invention thus may be formulated in conventional manner using one or more physiologically acceptable carriers comprising excipients and auxiliaries, which facilitate processing of the active compounds into preparations which, can be used pharmaceutically. Proper formulation is dependent upon the route of administration chosen.

Compounds that antagonize the adenosine receptor can be formulated as pharmaceutical compositions and administered to a mammalian subject, such as a human patient in a variety of forms adapted to the chosen route of administration, i.e., orally or parenteraUy, by intravenous, intramuscular, topical, transdermal or subcutaneous routes.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar or alginic acid or a salt thereof such as sodium alginate.

In addition, enteric coating may be useful as it is may be desirable to prevent exposure of the compounds of the invention to the gastric environment.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers.

In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For injection, the compounds of the invention may be formulated in aqueous solutions, preferably in physiologically compatible buffers such as Hank's or Ringer's solution or physiological saline buffer. For transmucosal and transdermal administration, penetrants appropriate to the barrier to be permeated may be used in the composition. Such penetrants, including for example DMSO or polyethylene glycol, are known in the art.

For administration by inhalation, the compounds for use according to the present invention are conveniently delivered in the form of an aerosol spray presentation from a pressurized pack or a nebulizer with the use of a suitable propellant, e. g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e. g., gelatin for use in an inhaler or insufflator may be formulated containing a powder mix of the compound and a suitable powder base such as lactose or starch.

Pharmaceutical compositions for parenteral administration include aqueous solutions of the active ingredients in water-soluble form. Additionally, suspensions of the active compounds may be prepared as appropriate oily injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents, which increase the solubility of the compounds, to allow for the preparation of highly concentrated solutions.

The compounds of the present invention may also be formulated in rectal compositions such as suppositories or retention enemas, using, e.g., conventional suppository bases such as cocoa butter or other glycerides.

Depending on the severity and responsiveness of the condition to be treated, dosing can also be a single administration of a slow release composition, with course of treatment lasting from several days to several weeks or until cure is effected or diminution of the disease state is achieved. The amount of a composition to be administered will of course, be dependent on many factors including the subject being treated, the severity of the affliction, the manner of administration, the judgment of the prescribing physician. The compounds of the invention may be administered orally or via injection at a dose from 0.001 to 2500 mg/kg per day. The dose range for adult humans is generally from 0.005 mg to 10 g/day. Tablets or other forms of presentation provided in discrete units may conveniently contain an amount of compound of the invention which is effective at such dosage or as a multiple of the same, for instance, units containing 5 mg to 500 mg, usually around 10 mg to 200 mg. The precise amount of compound administered to a patient will be the responsibility of the attendant physician. However, the dose employed will depend on a number of factors, including the age and sex of the patient, the precise disorder being treated, and its severity. Also, the route of administration may vary depending on the condition and its severity.

As used herein, and as would be understood by the person of skill in the art, the recitation of “a compound” is intended to include salts, solvates and inclusion complexes of that compound. The term “solvate” refers to a compound in the solid state, wherein molecules of a suitable solvent are incorporated in the crystal lattice. A suitable solvent for therapeutic administration is physiologically tolerable at the dosage administered. Examples of suitable solvents for therapeutic administration are ethanol and water. When water is the solvent, the solvate is referred to as a hydrate. In general, solvates are formed by dissolving the compound in the appropriate solvent and isolating the solvate by cooling or using an antisolvent. The solvate is typically dried or azeotroped under ambient conditions. Inclusion complexes are described in Remington: The Science and Practice of Pharmacy 19th Ed. (1995) volume 1, page 176-177, which is incorporated herein by reference. The most commonly employed inclusion complexes are those with cyclodextrins, and all cyclodextrin complexes, natural and synthetic, are specifically encompassed within the claims.

The term “pharmaceutically acceptable salt” refers to salts prepared from pharmaceutically acceptable non-toxic acids or bases including inorganic acids and bases and organic acids and bases. When the compounds of the present invention are basic, salts may be prepared from pharmaceutically acceptable non-toxic acids including inorganic and organic acids. Suitable pharmaceutically acceptable acid addition salts for the compounds of the present invention include acetic, benzenesulfonic (besylate), benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric acid, p-toluenesulfonic, and the like. When the compounds contain an acidic side chain, suitable pharmaceutically acceptable base addition salts for the compounds of the present invention include metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucarnine) and procaine.

The term “preventing” as used herein refers to administering a medicament beforehand to forestall or obtund an attack. The person of ordinary skill in the medical art (to which the present method claims are directed) recognizes that the term “prevent” is not an absolute term. In the medical art it is understood to refer to the prophylactic administration of a drug to substantially diminish the likelihood or seriousness of a condition, and this is the sense intended herein.

It should be understood that in addition to the ingredients particularly mentioned above, the formulations of this invention may include other agents conventional in the art having regard to the type of formulation in question, for example those suitable for oral administration may include flavoring agents.

The compositions may be presented in a packaging device or dispenser, which may contain one or more unit dosage forms containing the active ingredient. Examples of a packaging device include metal or plastic foil, such as a blister pack and a nebulizer for inhalation. The packaging device or dispenser may be accompanied by instructions for administration. Compositions comprising a compound of the present invention formulated in a compatible pharmaceutical carrier may also be placed in an appropriate container and labeled for treatment of an indicated condition.

The compounds and compositions of the present invention may be used as a stand alone treatment or administered in combination with additional agents useful in treating neurodegenerative disorders, movement disorders, depression, for example in combination with L-dopa.

Combination therapy can be achieved by administering two or more agents, each of which is formulated and administered separately, or by administering two or more agents in a single formulation. Other combinations are also encompassed by combination therapy. For example, two agents can be formulated together and administered in conjunction with a separate formulation containing a third agent. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within any number of hours of each other or within any number or days or weeks of each other. In some cases even longer intervals are possible.

While in many cases it is desirable that the two or more agents used in a combination therapy be present in within the patient's body at the same time, this need not be so. Combination therapy can also include two or more administrations of one or more of the agents used in the combination. For example, if agent X and agent Y are used in a combination, one could administer them sequentially in any combination one or more times, e.g., in the order X—Y—X, X—X—Y, Y—X—Y, Y—Y—X, X—X—Y—Y, etc.

As antagonists of A₂ₐ receptors, the compounds of formula I have utility in treating and preventing inter alia neurodegenerative disorders and depression. The compounds and compositions can be used advantageously in combination with other agents useful in treating neurodegenerative disorders and depression. For example, a compound or compounds of formula I may be used in preparing a composition further comprising L-dopa and or caffeine for utility in the treatment of Parkinson's and related diseases.

The compounds of the present invention are useful in inhibiting the activity of adenosine receptors or in inhibiting adenosine receptor-mediated activity and are useful in treating complications arising therefrom.

In some embodiments the compounds of the present invention are useful in inhibiting the activity of A₂ₐ receptors or in inhibiting A₂ₐ receptor-mediated activity and are useful in treating complications arising therefrom.

According to the present invention, the A₂ₐ receptor antagonists may be administered prophylactically, i.e., prior to onset of a neurological disorder, or they may be administered after onset of the disorder, or at both times.

A₂ₐ antagonists have been shown to produce an increase in locomotor activity, a decrease of neuroleptic-induced catalepsy, decrease of MPTP-induced hypomotility, reversal of cocaine withdrawal-induced anhedonia and several indications of neuroprotection in response to brain injury. These observations support therapeutic indications of A₂ₐ antagonists for inter alia Parkinson's disease (PD) and cocaine abuse, and neurodegenerative disorders such as Alzheimer's disease.

A₂ₐ antagonists, such as SCH 58261 and KW-6002, are particularly compelling for the treatment of PD since they not only enhance locomotor activity in animal models as a stand-alone treatment, but they potentiate the activity of L-dopa so that levels of L-dopa with reduced propensity to elicit dyskenesias can be given (Chen, Drug News Perspect. 2003, 16, 597; Morelli et al, Drug Dev. Res. 2001, 52, 387; Bara-Jimenez et al, Neurology 2003, 61, 293). Furthermore, the efficacy of A₂ₐ antagonists does not diminish upon repeated exposure, as seen for L-dopa (Halldner et al, Eur. J. Pharmacol. 2000, 406, 345). A distinct advantage of A₂ₐ antagonists over Ldopa is the propensity for neuroprotection (Morelli et al, Neurotox. Res. 2001, 3, 545). Although the compounds of the invention are selective A₂ₐ antagonists, some of them may exhibit sufficient residual affimity for other classes of adenosine receptors to be useful to treat conditions associated with additional adenosine receptors. As a result, the present invention also provides a method of treating a disorder associated with the A₂ₐ receptor and one or more of A₁, A_{2b} or A₃ receptors.

The adenosine receptor antagonists of the present invention are useful in effecting neuroprotection and in treating central nervous system and peripheral nervous system diseases, neurodegenerative diseases, cardiovascular diseases, cognitive disorders, CNS injury, renal ischemia; acute and chronic pain; affective disorders; cognitive disorders; central nervous system injury; cerebral ischemia; myocardial ischemia; muscle ischemia; sleep disorders; eye disorders and diabetic neuropathy;

In some embodiments the CNS and PNS disorders are movement disorders. A movement disorder may be selected from a disorder of the basal ganglia which results in dyskinesias. Non-limitative disorders include Huntington's disease, multiple system atrophy, progressive supernuclear palsy, essential tremor, myoclonus, corticobasal degeneration, Wilson's disease, progressive pallidal atrophy, Dopa-responsive dystoma-Parkinsonism, spasticity, Alzheimer's disease and Parkinson's disease.

Parkinson's disease further includes early-onset Parkinson's disease, drug-induced Parkinsonism, post-encephalitic Parkinsonism, Parkinsonism induced by poisoning and post-traumatic Parkinson's disease.

The compounds of the present invention have utility as neuroprotectants and may be useful in preventing or treating traumatic brain injury (TBI) and for the attenuation of cognitive impairment in coronary artery bypass graft (CABG) patients. As such the compounds and compositions may be administered to a subject at risk of neural ischemia.

The following examples will further describe the invention, and are used for the purposes of illustration only, and should not be considered as limiting the invention being disclosed.

Abbreviations: The following abbreviations and terms have the indicated meaning throughout, unless otherwise stated:
Ac—acetyl
AcOH—Acetic acid
Boc—teri-butoxycarbonyl
Boc₂O—tert-butoxycarbonic anhydride
Bu—butyl
C—carbon
c—cyclo
CDCl₃—Deuterated chloroform
CD₃OD—Deuterated methanol
δ—NMR chemical shift referenced to tetramethylsilane
DCE—1,2-dichloroethane
DCM—dichloromethane=methylene chloride=CH₂Cl₂
DIC—Diisopropyl carbodiimide
DIPEA—Diisopropylethylamine
DMA—N,N-dimethylacetamide
DMAP—4-Dimethylamino pyridine
DMF—N,N-dimethylformamide
DMSO—Dimethyl sulfoxide
EDC—N-(3-Dimethylaminopropyl)ethylcarbodiimide hydrochloride salt
Et—Ethyl
EtOAc—Ethyl acetate
ESI—Electrospray ionization
Et₃N—Triethylamine
Et₃SiH—Triethylsilane
¹H NMR—Proton Nuclear Magnetic Resonance
h—hours
Hexanes—HPLC grade isomeric hexanes
HOBt—hydroxybenzotriazole
i—iso
LCMS—Liquid Chromatography Mass Spectroscopy
m-—meta
Me—methyl
MeOH—methanol=CH₃OH
min—minutes
n—normal
N—nitrogen
NMR—Nuclear Magnetic Resonance
NaBH₄—sodium borohydride
NaCNBH₃—sodium cyano borohydride
Na(OAc)₃BH—sodium triacetoxy borohydride
o-—ortho
p-—para
Ph—Phenyl
r.t.—room temperature
sat.—saturated
s—secondary
t—tertiary
TFA—trifluoro acetic acid
THF—tetrahydrofuran

### EXAMPLE 1

### Synthesis of Aminopurine erivatives

Compounds of formula I can be synthesized by means of conventional organic synthesis executable by those skilled in the art. The illustration of examples, but not the limitation, of the synthesis of compounds of formula I is detailed in scheme 1, hereinbelow:

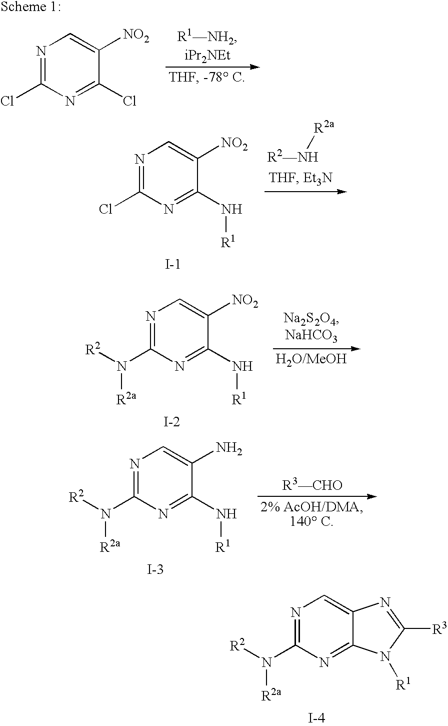

### Synthesis

Compounds of formula I can be synthesized in four steps from commercially available 2,4-dichloro-5-nitropyrimidine (Scheme 1). Initial N-arylation of a primary amine (R¹—NH₂) with 2,4-dichloro-5-nitropyrimidine provides a mixture (typically a 10:1 ratio) of regioisomers which can be readily separated by flash chromatography. The predominant regioisomer (corresponding to amino substitution at the C-4 position, I-1) is further functionalized at C-2 with a primary or secondary amine (R²—NH—R^{2a}) to afford I-2, and followed by nitro reduction to provide I-3. Purine formation is achieved by heating with an aldehyde in 2% v/v AcOH/DMA to afford I-4.

Analogous compounds of formula I can be synthesized using similar experimental procedures.

### Procedure A

### Intermediate 1 (I-1)-2-Chloro-N-(3-methoxypropyl)-5-nitropyrimidin-4-amine

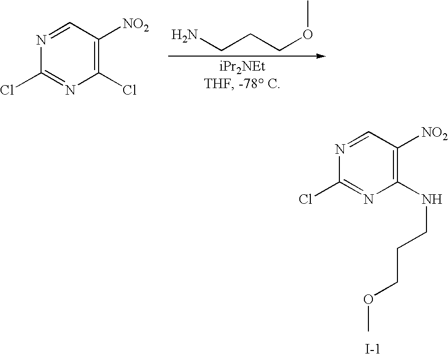

To 2.0 g (10.3 mmol, 1.0 eq.) of 2,4-dichloro-5-nitropyrimidine in 10 mL of THF at −78° C. under an argon atmosphere was added 3.9 mL (22.7 mmol 2.2 eq.) of N,N-diisopropylethylamine and 1.1 mL (10.3 mmol, 1.0 eq.) of 3-methoxypropyl amine. The reaction mixture was stirred for 30 min at −78° C. and then allowed to warm to 25° C. and stirred for an additional 1 h. The solvent was removed in vacuo and the residue partitioned between 100 mL of EtOAc and 50 mL of water. The organic solution was washed with 50 mL of saturated brine, dried (Na₂SO₄) and the solvent removed in vacuo. The product was purified by flash chromatography (10% EtOAc/hexanes) to provide 2.0 g (8.1 mmol, 79%) of 2-chloro-N-(3-methoxypropyl)-5-nitropyrimidin-4-amine (I-1) as a yellow solid. δ_{H} (300 MHz, CDCl₃) 1.96 (m, 2H), 3.43 (s, 3H), 3.61 (t, 2H), 3.78 (q, 2H), 9.03 (s, 1H), 9.17 (bs, 1H); m/z (ESI) found 247.1, [M+H]⁺.

### Procedure B

### Intermediate 3 (I-3)-N⁴-(3-Methoxypropyl)-N²-(2-(thiophen-2-yl)ethyl)pyrimidine-2,4,5-triamine

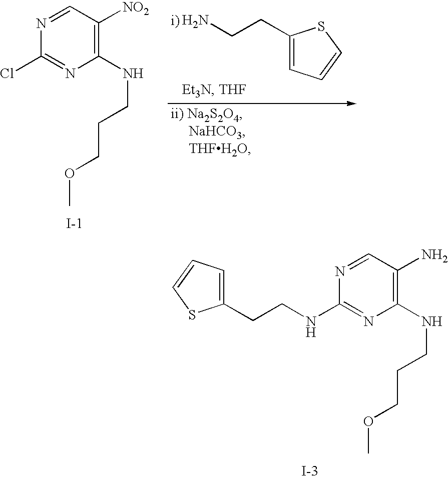

To a solution of 0.19 g (0.77 mmol, 1.0 eq.) of 2-chloro-N-(3-methoxypropyl)-5-nitropyrimidin-4-amine (I-1) in 5 mL of THF was added 0.22 mL (1.54 mmol, 2.0 eq.) of triethylamine and 0.09 mL (0.81 mmol, 1.05 eq.) of 2-thiophene ethylamine. The reaction mixture was stirred for at room temperature for 1 h and 2 mL of THF added. A solution of 0.6 g (˜3 mmol, ˜85% tech. grade, ˜4 eq) of sodium hydrosulfite and 0.30 g (3.6 mmol, ˜4.7 eq.) of sodium hydrogen carbonate in 5 mL of water was added and the mixture stirred vigorously at room temperature for 1 h. The mixture was diluted with 20 mL of EtOAc and washed with 15 mL of sat. NaHCO₃ (aq) and 10 mL of sat. brine. The organic phase was dried (Na₂SO₄) and the solvent removed in vacuo to provide 0.15 g of crude N⁴-(3-methoxypropyl)-N²-(2-(thiophen-2-yl)ethyl)pyrimidine-2,4,5-triamine (I-3). m/z (ESI) found 308.2 [M+H]⁺.

### Procedure C

### Intermediate 4 (I-4)-3-(9-(3-methoxypropyly-2-(2-(thiophen-2-yl)ethylamino)-9H-purin-8-yl)benzonitrile

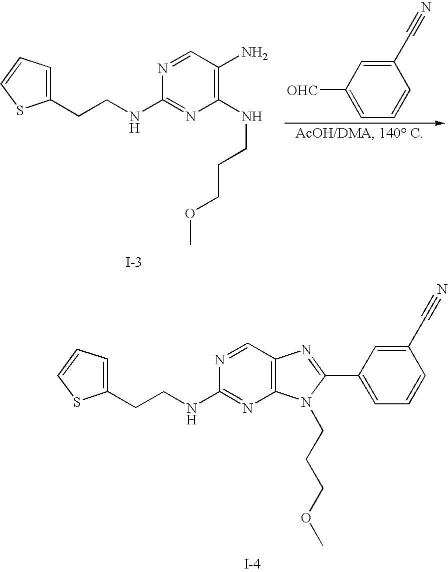

To a solution of 0.15 g (0.48 mmol, 1.0 eq.) of N⁴-(3-Methoxypropyl)-N²-(2-(thiophen-2-yl)ethyl)pyrimidine-2,4,5-triamine (I-3) in 3 mL of 2% v/v AcOH/DMA was added 0.13 g (0.97 mmol, 2.0 eq.) of 3-cyanobenzaldehyde. The mixture was stirred at 140° C. for 16 h. and the solvent removed in vaccuo. The residue purified by flash chromatography (50% EtOAc/hexanes) and the product triturated with diethyl ether to provide 0.06 g (0.14 mmol, 30%) of 3-(9-(3-methoxypropyl)-2-(2-(thiophen-2-yl)ethylamino)-9H-purin-8-yl)benzonitrile (I-4) as a yellow solid. δ_{H} (300 MHz, CDCl₃) 2.08 (m, 2H), 3.20 (t, 2H), 3.22 (s, 3H), 3.34 (t, 2H), 3.78 (q, 2H), 4.36 (t, 2H), 5.38 (bt, 1H), 6.89 (m, 1H), 6.97 (m, 1H), 7.18 (d, 1H), 7.66 (t, 1H), 7.80 (m, 1H), 8.06 (d, 1H), 8.16 (m, 1H), 8.72 (s, 1H); m/z (ESI) found 419.1 [M+H]⁺.

Analytical HPLC analysis:

Method A: Waters Millenium 2690/996PDA separations system employing a Phenomonex Luna 3u C8 50×4.6 mm analytical column. The aqueous acetonitrile based solvent gradient involves;

0-1 min-Isocratic 10% of (0. 1% TFA/acetonitrile); 1 min-7 min-Linear gradient of 10-90% of (0. l% TFA/acetonitrile): 7 min-9 min-Isocratic 90% of (0.1% TFA/acetonitrile); 9 min-10 min-Linear gradient of 90-10% of (0.1% TFA/acetonitrile); 10 min-12 min-Isocratic 10% of (0. 1% TFA/acetonitrile). Flow rate=1 mL/min.

Method B: Waters Millenium 2690/996PDA separations system employing a Phenomenex Columbus 5u c18 column 50×4.60 mm analytical column. The aqueous acetonitrile based solvent gradient involves;

0-0.5 min-Isocratic 10% of (0.05% TFA/acetonitrile); 0.5 min-5.5 min-Linear gradient of 10-90% of (0.05% TFA/acetonitrile): 5.5 min-7.5 min-Isocratic 90% of (0.05% TFA/acetonitrile); 7.5 min-8 min-Linear gradient of 90-10% of (0.05% TFA/acetonitrile); 8 min-10 min-Isocratic 10% of(0.05% TFA/acetonitrile). Flow rate=0.4 mL/min.

Mass Spectroscopy

Mass Spectroscopy was conducted using a Thermo-electron LCQ classic or an Applied Biosciences PE Sciex API150ex. Liquid Chromatography Mass Spectroscopy was conducted using a Waters Millenium 2690/996PDA linked Thermo-electron LCQ classic.

NMR Spectroscopy

¹H NMR spectroscopy was conducted using a Varian 300 MHz Gemini 2000 FTNMR.

A₂ₐ Binding Assay:

Membranes prepared from HEK-293 cells that express human A₂ₐ (0.04 mg/mL final, PerkinElmer Life and Analytical Sciences, Boston, Mass.) were mixed with yttrium oxide wheatgerm-agglutinin (WGA)-coated SPA beads (4 mg/mL final, Amersham Biosciences, Piscataway, N.J.) and adenosine deaminase (0.01 mg/ml final) in assay buffer (Dulbecco's phosphate-buffered saline containing 10 mM MgCl₂) for 15 minutes at 4° C. This mixture (10 μL) was added with agitation to the test compounds (10 μL) prepared in 2.5% DMSO or to 2.5% DMSO (1% final) in 384-well assay plates (Corning #3710).

Binding was initiated with the addition of 5 μL of [³H]SCH 58261 (2 nM final, Amersham Biosciences) immediately followed by centrifugation at 1000 rpm for 2 min. The assay plates were incubated in the dark, overnight at room temperature and the signal was detected using a ViewLux CCD Imager (PerkinElmer). Compounds were tested at 11 different concentrations ranging from 0.1 nM to 10 μM Nonspecific binding was determined in the presence of 10 μM CGS 15943. Assays were performed in duplicate and compounds were tested at least twice. The data were fit to a one-site competition binding model for IC₅₀ determination using the program GraphPad Prism (GraphPad Software, Inc., San Diego, Calif.) and Ki values were calculated using the Cheng-Prusoff equation (Cheng, Y, Prusoff, W. H. Biochem. Pharmacol. 1973, 22, 3099). The IC₅₀ s of all the compounds in the table below were less than 10 μM.

Aₐ Binding Assay:

As described in Matasi et al. (Bioorg. Med. Chem. Lett. 2005, 15, 1333), membranes (10 μg) prepared from CHO (Chinese Hamster Ovary) cells that express human A₁ were mixed with 1 nM (final) [³H]DPCPX in 200 μL assay buffer (2.7 mM KCl, 1.1 mM KH₂PO₄, 137 mM NaCl, 7.6 mM Na₂HPO4, 10 mM MgCl₂, 0.04% methyl cellulose, 20 ug/mL adenosine deaminase) containing 4% DMSO with or without test compounds. Reactions were carried out for 60 min at room temperature and were terminated by rapid filtration over GF/B filters. Filters were washed seven times with 1 mL cold distilled H₂O, air dried, and radioactivity retained on filters were counted in a Packard TopCount® NXT microplate scintillation counter (Global Medical Instrumentation, Inc., Ramsey, Minn.). Compounds were tested at 10 different concentrations ranging from 0.1 nM to 10 μM. Nonspecific binding was determined in the presence of 10 uM NECA (5′-(N-Ethylcarboxamido)adenosine). Assays were preformed in duplicate and compounds were tested two times. Data were fit to a one-site competition binding model for IC₅₀ determination using the program GraphPad Prism (GraphPad Software, Inc., San Diego, Calif.) and Kᵢ values were calculated using the Cheng-Prusoff equation (Cheng, Y, Prusoff, W. H. Biochem. Pharmacol. 1973, 22, 3099).

Representative examples are shown in table 1.

TABLE 1

| Example | Structure | hplc (min)/Method | m/z [M + H]+ |
|---|---|---|---|
| 1 | 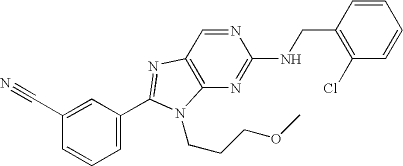 | 6.1 min Method A | 433.1 |
| 2 | 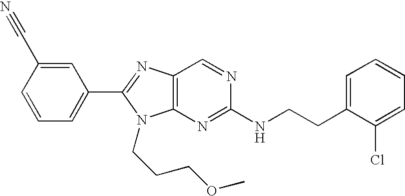 | 6.2 min Method A | 447.2 |
| 3 | 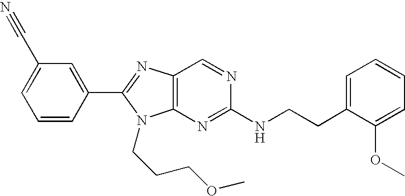 | 5.9 min Method B | 443.2 |
| 4 | 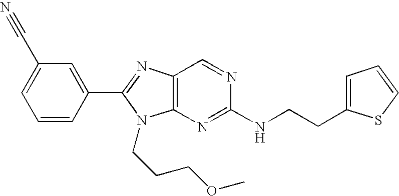 | 5.8 min Method A | 419.1 |
| 5 | 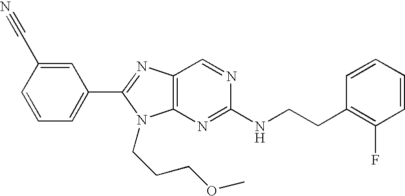 | 6.1 min Method A | 431.2 |
| 6 | 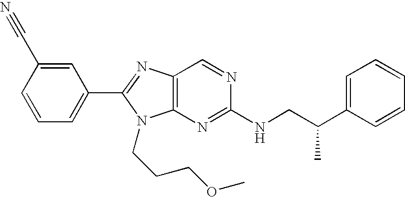 | 6.1 min Method A | 427.2 |
| 7 | 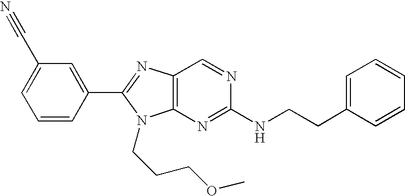 | 5.9 min Method B | 413.2 |
| 8 | 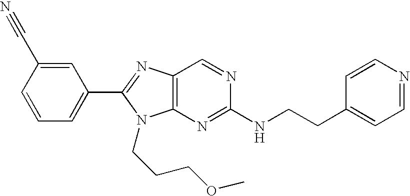 | 4.1 min Method A | 414 |
| 9 | 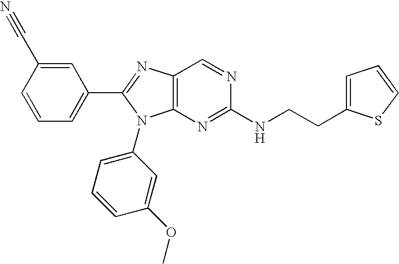 | 6.5 min Method A | 453.1 |
| 10 | 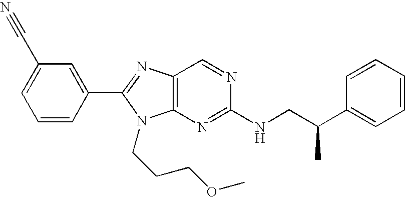 | 6.1 min Method A | 427.2 |
| 11 | 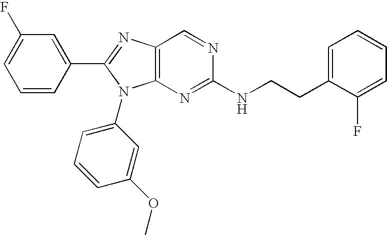 | 6.8 min Method A | 458.1 |
| 12 | 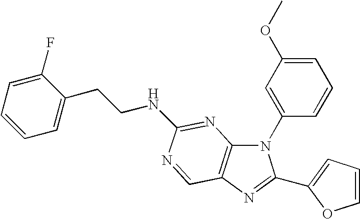 | 6.8 min Method A | 430.1 |
| 13 | 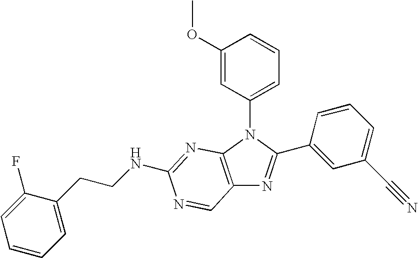 | 6.6 min Method A | 465.1 |
| 14 | 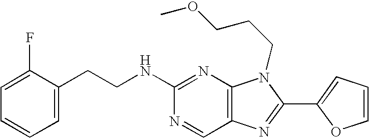 | 5.7 min Method B | 396.2 |
| 15 | 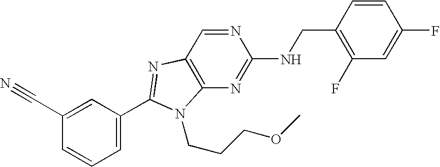 | 6.0 min Method A | 435.2 |
| 16 | 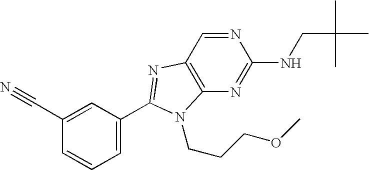 | 5.8 min Method A | 379.3 |
| 17 | 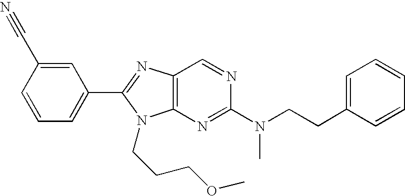 | 6.2 min Method A | 427.1 |
| 18 | 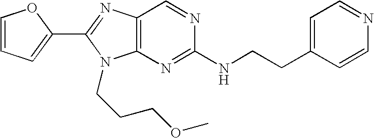 | 3.8 min Method A | 379.2 |
| 19 | 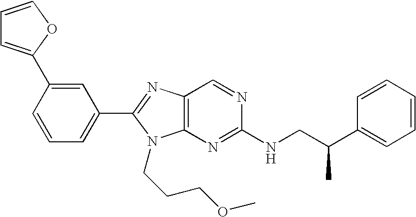 | 7.2 min Method B | 468.2 |
| 20 | 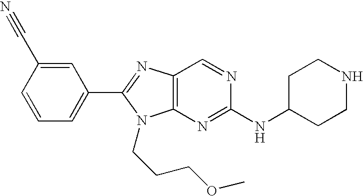 | 4.2 min Method B | 392.2 |

### 3-(2-(2-chlorobenzylamino)-9-(3-methoxypropyl)-9H-purin-8-yl)benzonitrle

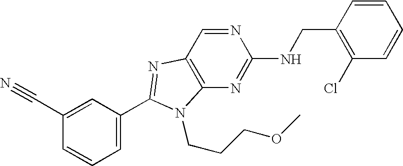 (δ_{H}, 300 MHz, CDCl₃) 1.90 (m, 2H), 3.02 (s, 3H), 3.13 (t, 2H), 4.20 (t 2H), 4.71 (d, 2H), 7.08 (m, 2H), 7.24 (m, 1H), 7.31 (m, 1H), 7.58 (m, 1H), 7.76 (m, 1H), 7.89 (m, 1H), 7.97 (m, 1H), 8.56 (s, 1H), 9.78 (bs, 1H); ESI, 433.1 [M+H].

### 3-(2-(2-chlorophenethylamino)-9-(3-methoxypropyl)-9H-purin-8-yl)benzonitrile

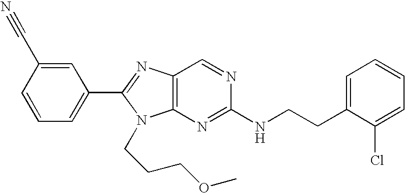 (δ_{H}, 300 MHz, CDCl₃) 1.94 (m, 2H), 3.00 (t, 2H), 3.08 (s, 3H), 3.22 (t, 2H), 3.70 (m, 2H), 4.22 (t, 2H), 7.01 (m, 2H), 7.17 (m, 2H), 7.58 (m, 1H), 7.75 (m, 1H), 7.92 (m, 1H), 8.01 (m, 1H), 8.41 (s, 1H), 9.55 (bs, 1H); ESI, 447.2 [M+H].

### 3-(2-(2-methoxyphenethylamino)-9-(3-methoxypropyl)-9H-purin-8-yl)benzonitrile

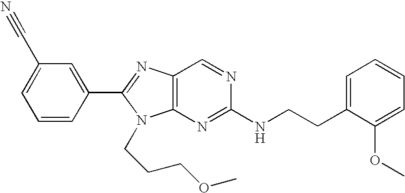 (δ_{H}, 300 MHz, CDCl₃) 1.96 (m, 2H), 2.90 (t, 2H), 3.07 (s, 3H), 3.20 (t, 2H), 3.62 (m, 2H), 3.69 (s, 3H), 4.22 (t, 2H), 6.72 (m, 2H), 7.06 (m, 2H), 7.58 (m, 1H), 7.75 (m, 1H), 7.93 (m, 1H), 8.01 (m, 1H), 8.31 (s, 1H), 9.68 (bs, 1H); ESI, 443.2 [M+H].

### 3-(9-(3-methoxypropyl)-2-(2-(thiophen-2-yl)ethylamino)-9H-purin-8-yl)benzonitrile

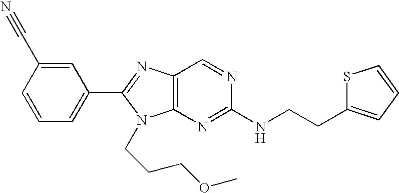 (δ_{H}, 300 MHz, CDCl₃) 2.08 (m, 2H), 3.20 (t, 2H), 3.22 (s, 3H), 3.34 (t, 2H), 3.78 (q, 2H), 4.36 (t, 2H), 5.38 (bt, 1H), 6.89 (m, 1H), 6.97 (m, 1H), 7.18 (d, 1H), 7.66 (t, 1H), 7.80 (m, 1H), 8.06 (d, 1H), 8.16 (m, 1H), 8.72 (s, 1H); ESI, 419.1 [M+H].

### 3-(9-(3-methoxypropyl)-2-((S)-2-phenylpropylamino)-9H-purin-8-yl)benzonitrile

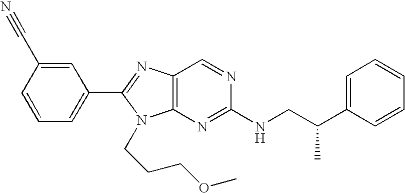 (δ_{H}, 300 MHz, CDCl₃) 1.26 (d, 3H), 1.95 (m, 2H), 3.01 (m, 1H), 3.08 (s, 3H), 3.22 (t, 2H), 3.60 (m, 2H), 4.21 (t, 2H), 7.12 (m, 5H), 7.58 (m, 1H), 7.76 (m, 1H), 7.92 (m, 1H), 8.00 (m, 1H), 8.38 (s, 1H), 9.54 (bs, 1H); ESI, 427.2 [M+H].

### 3-(9-(3-methoxypropyl)-2-(phenethylamino)-9H-purin-8-yl)benzonitrile

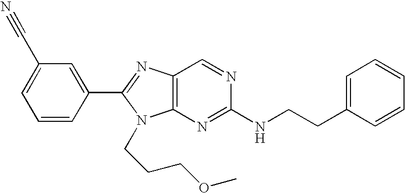 (δ_{H}, 300 MHz, CDCl₃) 2.11 (m, 2H), 3.03 (t, 2H), 3.23 (s, 3H), 3.36 (t, 2H), 3.80 (m, 2H), 4.38 (t, 2H), 7.26 (m, 5H), 7.74 (m, 1H), 7.90 (m, 1H), 8.08 (m, 1H), 8.16 (m, 1H), 8.54 (s, 1H), 9.85 (bs, 1H); ESI, 413.2 [M+H].

### 3-(9-(3-methoxypropyl)-2-(2-(pyridin-4-yl)ethylamino)-9H-purin-8-yl)benzonitrile

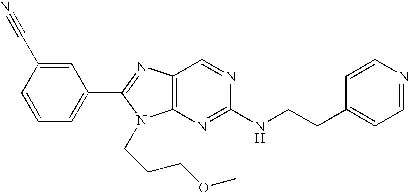 (δ_{H}, 300 MHz, CDCl₃) 2.12 (m, 2H), 2.95 (t, 2H), 3.20 (s, 3H), 3.30 (t, 2H), 3.75 (q, 2H), 4.34 (t, 2H), 5.41 (bt, 1H), 7.18 (d, 2H), 7.63 (t, 1H), 7.78 (m, 1H), 8.03 (m, 1H), 8.12 (m, 1H), 8.51 (d, 2H), 8.68 (s, 1H); ESI, 414.0 [M+H].

### 3-(9-(3-methoxyphenyl)-2-(2-(thiophen-2-yl)ethylamino)-9H-purin-8-yl)benzonitrile

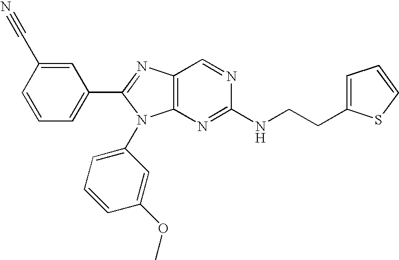 (δ_{H}, 300 MHz, CDCl₃) 3.13 (t, 2H), 3.68 (q, 2H), 3.80 (s, 3H), 5.68 (bs, 1H), 6.82 (m, 2H), 6.92 (m, 2H), 7.04 (m, 1H), 7.14 (dd, 1H), 7.40 (m, 2H), 7.65 (m, 1H), 7.75 (m, 1H), 7.88 (m, 1H), 8.79 (s, 1H); ESI, 453.1 [M+H].

### 3-(9-(3-methoxypropyl)-2-((R)-2-phenylpropylamino)-9H-purin-8-yl)benzonitrile

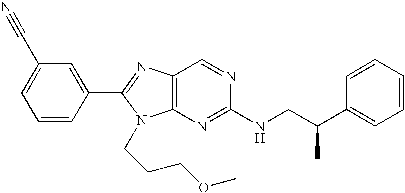 (δ_{H}, 300 MHz, CDCl₃) 1.24 (d, 3H), 1.96 (m, 2H), 3.01 (m, 1H), 3.08 (s, 3H), 3.22 (t, 2H), 3.60 (m, 2H), 4.21 (t, 2H), 7.12 (m, 5H), 7.60 (m, 1H), 7.78 (m, 1H), 7.91 (m, 1H), 7.99 (m, 1H), 8.48 (s, 1H), 9.15 (bs, 1H); ESI, 427.2 [M+H].

### N-(2-fluorophenethyl)-8-(3-fluorophenyl)-9-(3-methoxyphenyl)-9H-purin-2-amine

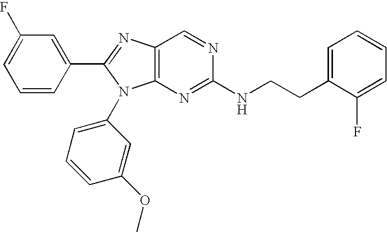 (δ_{H}, 300 MHz, CDCl₃) 2.95 (t, 2H), 3.65 (q, 2H), 3.82 (s, 3H), 6.85 (m, 2H), 6.90-7.35 (m, 9H), 7.43 (m, 1H), 8.55 (s, 1H); ESI, 458.1 [M+H].

### N-(2-fluorophenethyl)-8-(furan-2-yl)-9-(3-methoxyphenyl)-9H-purin-2-amine

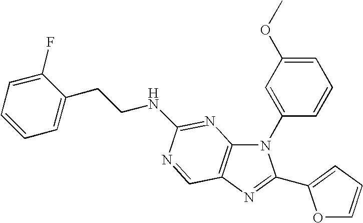 (δ_{H}, 300 MHz, CDCl₃) 2.78 (t, 2H), 3.45 (q, 2H), 3.72 (s, 3H), 6.12 (m, 1H), 6.30 (m, 1H), 6.75-7.20 (m, 7H), 7.40 (m, 1H), 7.46 (m, 1H), 7.67 (m, 1H), 7.76 (m, 1H), 7.88 (m, 1H), 8.28 (s, 1H), 9.90 (bs, 1H); ESI, 430.1 [M+H].

### 3-(2-(2-fluorophenethylamino)-9-(3-methoxyphenyl)-9H-purin-8-yl)benzonitrile

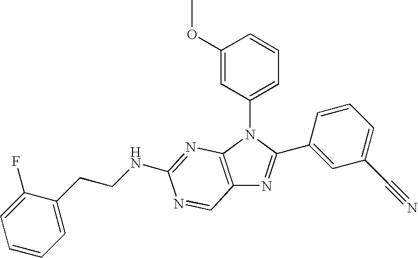 (δ_{H}, 300 MHz, CDCl₃) 2.95 (t, 2H), 3.67 (q, 2H), 3.82 (s, 3H), 5.60 (bs, 1H), 6.85 (m, 1H), 6.91 (m, 1H), 7.04 (m, 3H), 7.16 (m, 2H), 7.43 (m, 2H), 7.67 (m, 1H), 7.76 (m, 1H), 7.88 (m, 1H), 8.79 (s, 1H); ESI, 465.1 [M+H].

### N-(2-fluorophenethyl)-8-(furan-2-yl)-9-(3-methoxypropyl)-9H-purin-2-amine

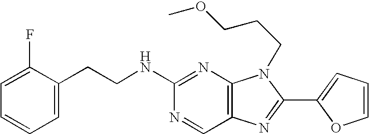 (δ_{H}, 300 MHz, CDCl₃) 1.97 (m, 2H), 2.92 (t, 2H), 3.10 (s, 3H), 3.32 (t, 2H), 3.64 (m, 2H), 4.51 (t, 2H), 6.55 (m, 1H), 6.90 (m, 2H), 7.20 (m, 2H), 7.28 (d, 2H), 7.58 (m, 1H), 8.21 (s, 1H), 9.90 (bs, 1H); ESI, 396.2 [M+H].

### 3-(2-(2,4-difluorobenzylamino)-9-(3-methoxypropyl)-9H-purin-8-yl)benzonitrile

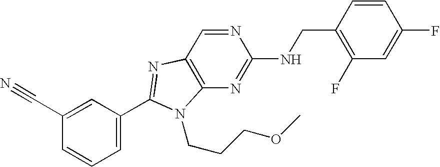 (δ_{H}, 300 MHz, CDCl₃) 1.89 (m, 2H), 3.06 (s, 3H), 3.18 (t, 2H), 4.21 (t, 2H), 4.58 (d, 2H), 6.70 (m, 2H), 7.30 (m, 1H), 7.57 (m, 1H), 7.74 (d, 1H), 7.91 (d, 1H), 7.99 (s, 1H), 8.41 (s, 1H), 10.20 (bs, 1H); ESI, 435.2 [M+H].

### 3-(9-(3-methoxypropyl)-2-(neopentylamino)-9H-purin-8-yl)benzonitrile

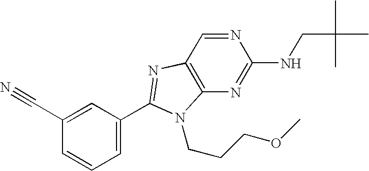 (δ_{H}, 300 MHz, CDCl₃) 0.88 (s, 9H), 1.97 (t, 2H), 3.08 (s, 3H), 3.24 (m, 4H), 4.23 (t, 2H), 7.58 (m, 1H), 7.74 (m, 1H), 7.93 (m, 1H), 8.02 (m, 1H), 8.31 (m, 1H), 9.56 (bs, 1H); ESI, 397.3 [M+H].

### 3-(9-(3-methoxypropyl)-2-(methyl(phenethyl)amino)-9H-purin-8-yl)benzonitrile

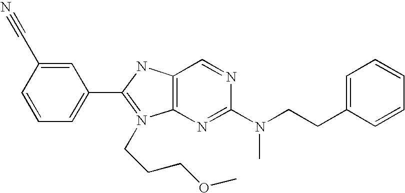 (δ_{H}, 300 MHz, CDCl₃) 2.18 (m, 2H), 2.98 (m, 2H), 3.20 (s, 3H), 3.24 (s, 3H), 3.35 (t, 2H), 3.93 (t, 2H), 4.36 (t, 2H), 7.30 (m, 5H), 7.64 (m, 1H), 7.78 (m, 1H), 8.08 (m, 1H), 8.16 (m, 1H), 8.78 (s, 1H); ESI, 427.1 [M+H].

### 8-(furan-2-yl)-9-(3-methoxypropyl)-N-(2-(pyridin-4-yl)ethyl)-9H-purin-2-amine

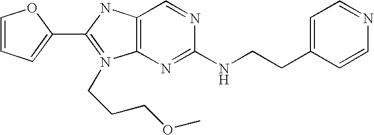 (δ_{H}, 300 MHz, CDCl₃) 2.08 (m, 2H), 2.96 (t, 2H), 3.24 (s, 3H), 3.38 (t, 2H), 3.75 (q, 2H), 4.52 (t, 2H), 5.22 (bt, 1H), 6.59 (m, 1H), 7.15 (m, 3H), 7.60 (m, 1H), 8.50 (m, 2H), 8.62 (s, 1H); ESI, 379.2 [M+H].

### 8-(3-(furan-2-yl)phenyl)-9-(3-methoxypropyl)-N-((R)-2-phenylpropyl)-9H-purin-2-amine

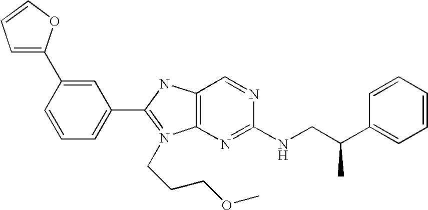 (δ_{H}, 300 MHz, CDCl₃) 1.23 (d, 3H), 1.96 (m, 2H), 3.00 (m, 1H), 3.03 (s, 3H), 3.18 (t, 2H), 3.50 (m, 2H), 4.23 (t, 2H), 6.36 (m, 1H), 6.63 (d, 1H), 7.05-7.20 (m, 5H), 7.39 (m, 2H), 7.47 (m, 1H), 7.67 (d, 2H), 7.91 (m, 1H), 8.50 (s, 1H); ESI, 468.2 [M+H].

Although the foregoing invention has been described in some detail for purposes of illustration, it will be readily apparent to one skilled in the art that changes and modifications may be made without departing from the scope of the invention described herein.

1. A compound according to formula I: 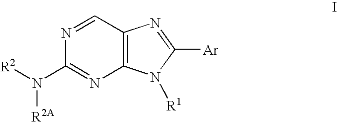
wherein
R¹ is a C₃-C₂₀ hydrocarbon in which at least one —CH₂— has been replaced by —O—;
R² is selected from the group consisting of H and lower alkyl;
R^{2A} is selected from the group consisting of C₁-C₂₀ hydrocarbon, heterocyclyl, heterocyclylalkyl, substituted alkyl, substituted arylalkyl and substituted heterocyclylalkyl;
Ar represents aryl, heteroaryl, substituted aryl and substituted heteroaryl.

2. A compound according to claim 1 wherein R¹ is selected from the group consisting of alkoxyalkyl, alkoxyaryl, alkoxyarylalkyl and oxygen heterocycle.

3. A compound according to claim 1 wherein R¹ is alkoxyalkyl.

4. A compound according to claim 3 wherein R¹ is methoxypropyl, of formula:

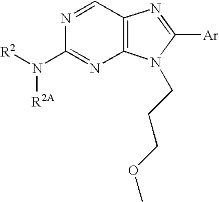

5. A compound according to claim 2 wherein R¹ is methoxyphenyl.

6. A compound according to claim 1 wherein R² is H.

7. A compound according to claim 1 wherein R^{2A} is C₁-C₂₀ hydrocarbon.

8. A compound according to claim 1 of formula 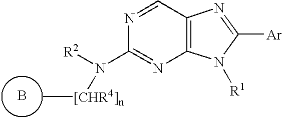
wherein
B is an aryl or heteroaryl ring, optionally substituted;
R⁴ is, in each of its occurrences independently H or methyl; and
n is 1 to 4.

9. A compound according to claim 7 wherein n is 1 or 2 and B is chosen from phenyl; phenyl substituted with halogen, methoxy, methyl or trifluoromethyl; thienyl, furanyl and pyridinyl.

10. A compound according to claim 1 wherein Ar is chosen from phenyl; thienyl; furanyl; and phenyl substituted with cyano, halogen, methoxy, methyl or trifluoromethyl.

11. A compound according to claim 9 wherein Ar is chosen from phenyl; thienyl; furanyl; and phenyl substituted with cyano, halogen, methoxy, methyl or trifluoromethyl.

12. A compound according to claim 11 wherein R¹ is alkoxyalkyl and R² is H.

13. A pharmaceutical composition comprising a pharmaceutically acceptable carrier and a therapeutically effective amount of at least one compound according to claim 1.

14. A composition according to claim 13 further comprising a second active ingredient selected from the group consisting of: (1) an agent useful in the treatment of Parkinson's disease, (2) an agent useful in the treatment of movement disorders, and (3) an agent useful in the treatment of depression.

15. A composition according to claim 14 wherein said second active ingredient is a dopaminergic receptor agonist.

16. A method of treating a disorder which is mediated by adenosine receptor function, which comprises administering to a subject in need of such treatment a therapeutically effective amount of a compound according to claim 1.

17. A method according to claim 16 wherein the disorder is a disorder associated with adenosine A₂ₐ receptors.

18. A method according to claim 16 wherein the disorder is selected from the group consisting of central nervous system and peripheral nervous system diseases; neurodegenerative diseases; cardiovascular diseases; cognitive disorders; CNS injury; renal ischemia; acute and chronic pain; affective disorders; cognitive disorders; central nervous system injury; cerebral ischemia; myocardial ischemia; muscle ischemia; sleep disorders; eye disorders and diabetic neuropathy.

19. A method according to claim 18 wherein the CNS and PNS disorders are movement disorders.

20. A method according to claim 19 wherein the movement disorder is selected from the group consisting of (1) diskinetic disorders of the basal ganglia; (2) Huntington's disease, (3) multiple system atrophy, (4) progressive supernuclear palsy, (5) essential tremor, (6) myoclonus, (7) corticobasal degeneration, (8) Wilson's disease, (9) progressive pallidal atrophy, (10) Dopa-responsive dystoma-Parkinsonism, (11) spasticity, (12) Alzheimer's disease and (13) Parkinson's disease.

21 A method according to claim 20 wherein the movement disorder is Parkinson's disease.

22. A method according to claim 16 wherein said method is for neuroprotection in a subject at risk of neural ischemia.

23. A method according to claim 16 wherein said method is for treating of injuries to the central nervous system.

24. A method according to claim 16 for treating restless leg syndrome.
